(19) United States
(12) Patent Application Publication
(10) Pub. No.: US 2007/0166349 A1
(43) Pub. Date: Jul. 19, 2007

(54) ORTHOPAEDIC IMPLANTS FABRICATED FROM AMORPHOUS OR PARTIALLY AMORPHOUS CALCIUM-BASED METAL ALLOYS

(76) Inventor: Dawn White, Ann Arbor, MI (US)

Correspondence Address: GIFFORD, KRASS, SPRINKLE,ANDERSON & CITKOWSKI, P.C, PO BOX 7021, TROY, MI, 48007-7021, US

(21) Appl. No.: 11/623,189

(22) Filed: Jan. 15, 2007

Related U.S. Application Data
(60) Provisional application No. 60/759,152, filed on Jan. 13, 2006.

(51) Int. Cl. A61F 2/28; B22F 1/02

(52) U.S. Cl. 424/423; 623/16.11; 419/38

(57) ABSTRACT

Orthopaedic implants are formed using processes whereby a metal alloy is cooled at a rate rapid enough that an amorphous or partially amorphous structure is retained. In the preferred embodiment, the metal alloy is a calcium-based metal alloy. The fabrication process may include die-casting or additive manufacturing process of the type wherein material increments are consolidated in accordance with the description without melting the material in bulk. Such processes include ultrasonic consolidation, electrical resistance consolidation, and frictional consolidation. The material increments are provided in the form of sheets, elongated tapes, filaments, dots or droplets. A preferred method includes a casting process to produce an initial form having an outer surface followed by an additive manufacturing process used to build up at least a portion of the outer surface. For example, the portion may include an intramedullary stem, bone-ingrowth surface, or articulating surface.

### REFERENCE TO RELATED APPLICATION

This application claim priority from U.S. Provisional Patent Application Ser. No. 60/759,152, filed Jan. 13, 2006, the entire content of which is incorporated herein by reference.

### FIELD OF THE INVENTION

This invention relates generally to orthopedic implants and, in particular, to implants formed using processes whereby a metal alloy is cooled at a rate rapid enough that an amorphous or partially amorphous structure is retained.

### BACKGROUND OF THE INVENTION

Orthopedic implants for replacing broken or diseased bones or teeth are typically based on special metals or ceramics having inert surface characteristics, and mechanical properties suitable for load-bearing applications in the bones or joints of interest. Because the body is highly corrosive to many metals, corrosion resistant materials such as stainless steels, titanium based alloys, and cobalt based alloys are typically employed. However, the lack of chemical interaction between implant and bone can lead to a weak bond between the implant and the underlying bone, which may result in aseptic loosening within only 5-10 years. The rate of implant loosening can be slowed by mechanically roughening the surface of the implant to provide features onto which the bone can attach.

Another approach to the problem is to involve the use of resorbable materials, typically calcium based, such as hydroxyapatite, which is sometimes applied as a coating to a metal implant, or used as an implant material. However, since hydroxyapatites are fairly weak their usefulness is limited. The promotion of bone growth on the surface of implants using coatings of bioactive glass or hydroxyapatite is expensive, and the coatings are brittle. Mechanical methods of promoting bone to implant adhesion have only a temporary effect, as the bone does not grow onto the implant. These drawbacks limit the working life of current implants, which requires additional operations to fit new implants when the old ones loosen.

This concept has been expanded to bioactive glasses such as machineable glass-ceramics, and dense hydroxylapatite bioactive composites such as polyethylene-hydroxylapatite. All of the above bioactive materials form an interfacial bond with bone. Bioactive glass is characterized by its ability to attach firmly to living tissue. It can, for example, guide tissue growth and bond chemically with bone. Tissue bonds to bioactive class due to formation of a silica gel (Si-gel) layer on the glass. The silica-rich layer acts as a template for a calcium phosphate precipitation which then bonds to the bone. This makes bioactive glass a unique material for filling defects and replacing damaged bony tissue Bioactive properties are mainly found in glasses with SiO₂—Na₂O/K₂O—CaO/MgO—B₂O₃—P₂O₅ compositions.

### SUMMARY OF THE INVENTION

This invention resides in methods of fabricating orthopaedic implants, and implants formed thereby, using processes whereby a metal alloy is cooled at a rate rapid enough that an amorphous or partially amorphous structure is retained. In the preferred embodiment, the metal alloy is a calcium-based metal alloy. The fabrication process may include die-casting or additive manufacturing process of the type wherein material increments are consolidated in accordance with the description without melting the material in bulk. Such processes include ultrasonic consolidation, electrical resistance consolidation, and frictional consolidation. The material increments are provided in the form of sheets, elongated tapes, filaments, dots or droplets.

A preferred method includes a casting process to produce an initial form having an outer surface followed by an additive manufacturing process used to build up at least a portion of the outer surface. For example, the portion may include an intramedullary stem, bone-ingrowth surface, or articulating surface.

### BRIEF DESCRIPTION OF THE DRAWINGS

FIG. 1 is a simplified block diagram of an ultrasonic consolidation system.

### DETAILED DESCRIPTION OF THE INVENTION

This invention resides in novel alternatives to the use of amorphous metal alloys produced from bio-compatible and/or bio-active materials such as calcium. In the preferred embodiments, medical implants are fabricated using calcium-based metal alloys in the amorphous, or glassy state. While calcium typically corrodes very rapidly, certain calcium alloys form amorphous structures very readily, and these structures are both far stronger and more corrosion resistant than crystalline calcium structures. Such materials may provide improved performance over either bio-active ceramics such as hydroxyapatites, or corrosion-resistant structural metals of the type currently in widespread use. It is possible in accordance with the invention to produce alloys that are both strong and resorbable, allowing bone growth into the implant, and a strong implant during the period of bone redevelopment.

Amorphous metals typically require very high cooling rates to produce. As a result, the materials are often available only as foils, ribbons, and powders. According to this invention, however, orthopaedic implants may be die cast or produced using additive manufacturing techniques. As a further alternative, forms produced through die casting or other processes may be at least partially coated with a metal alloy in an amorphous or partially amorphous state using an additive manufacturing process or other technique. The invention is applicable to all type of implants, including joints, such as the hip, knee, shoulder, elbow, wrist, ankle, hands and feet, vertebrae, and well as non-joint bone segments and teeth.

According to the invention, die casting of calcium-based metal alloys may be used in conjunction with high cooling rates, such that an amorphous structure is retained. The alloying elements are selected, and the final composition is determined, to provide an amorphous structure with improved corrosion resistance, strength, and bio-compatibility/resorbability. The amorphous or glassy state may be partially transformed to a crystalline state during manufacturing of the implant geometry in order to control the mechanical properties such as ductility, toughness, or other desired characteristics of final article. Then again, the production of implants from such precursor materials may require the use of additive manufacturing techniques to produce the final part geometries desired for medical applications.

Ultrasonic consolidation is a solid-state, low-temperature additive manufacturing process that can retain an amorphous structure while allowing implants or arbitrary geometry to be produced from these featureless feedstocks fabricated using very high cooling rates. A three-dimensional object is formed by consolidating material increments in accordance with a description of the object using a process that produces an atomically clean faying surface between the increments without melting the material in bulk.

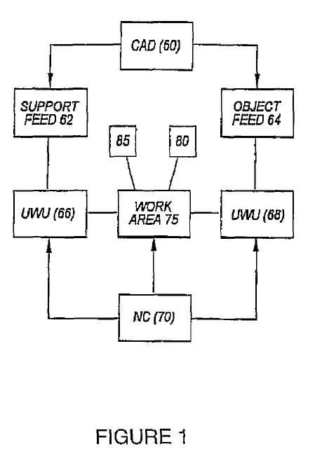

Referring to FIG. 1, a CAD system (60) interfaces with a numerical controller (70), which controls an actuation system. The actuation system brings the support feed unit (62) the support ultrasonic welding unit (66), the object feed unit (64) and the object ultrasonic welding unit (68) into proper position in the work area (75), so that the ultrasonic consolidation of the layers takes place according to the CAD description of the object. In alternative embodiments, electrical resistance, and frictional methodologies are used for object consolidation. These processes are described in detail in one or more of the following commonly assigned U.S. patents, the entire content of each being incorporated herein by reference: U.S. Pat. Nos. 6,814,823; 6,519,500; 6,463,349; 6,457,629; 6,443,345.

Using additive manufacturing, amorphous feedstocks are deposited and bonded together at low temperature, in the solid state, using ultrasonic, resistance, kinetic spray, or friction bonding or other solid-state processes to produce the desired implant geometry. In alternative embodiments, the amorphous feedstocks are deposited and bonded together using processes that support very high local cooling rates such as laser deposition, liquid droplet directed sprays or other liquid-phase techniques to produce the desired geometry.

I claim:

1. A method of fabricating an orthopaedic implant, comprising the steps of:
providing a description of an orthopaedic implant to be fabricated; and
forming the orthopaedic implant in accordance with the description using a metal alloy and a process having a cooling rate sufficiently rapid to retain an amorphous structure.

2. The method of claim 1, wherein the metal alloy is a calcium-based metal alloy.

3. The method of claim 1, wherein the process includes die-casting.

4. The method of claim 1, wherein the process includes an additive manufacturing process of the type wherein material increments are consolidated in accordance with the description without melting the material in bulk.

5. The method of claim 4, wherein the process is based upon ultrasonic consolidation.

6. The method of claim 4, wherein the process is based upon electrical resistance consolidation.

7. The method of claim 4, wherein the process is based upon frictional consolidation.

8. The method of claim 4, wherein the increments are provided in the form of sheets, elongated tapes, filaments, dots or droplets.

9. The method of claim 1, wherein the process includes.
a casting process to produce an initial form having an outer surface; and
an additive manufacturing process to build up at least a portion of the outer surface, the additive manufacturing process being of the type wherein material increments are consolidated in accordance with the description without melting the material in bulk.

10. The method of claim 9, wherein the process is based upon ultrasonic consolidation.

11. The method of claim 9, wherein the process is based upon electrical resistance consolidation.

12. The method of claim 9, wherein the process is based upon frictional consolidation.

13. The method of claim 9, wherein the increments are provided in the form of sheets, elongated tapes, filaments, dots or droplets.

14. An orthopaedic implant fabricated according to the process of claim 1.

14. An orthopaedic implant fabricated according to the process of claim 4.

14. An orthopaedic implant fabricated according to the process of claim 9.

15. An orthopaedic implant, at least a portion of which is composed of a metal alloy in an amorphous or partially amorphous state.

16. The implant of claim 15, wherein the metal alloy is a calcium-based alloy.

17. The implant of claim 15, wherein the implant is joint-related.

18. The implant of claim 15, wherein the portion includes an intramedullary stem, bone-ingrowth surface, or articulating surface.

19. The implant of claim 15, wherein the metal alloy in an amorphous or partially amorphous state is produced through die casting.

20. The implant of claim 15, wherein the metal alloy in an amorphous or partially amorphous state is produced through additive manufacturing.
